(19) United States
(12) Patent Application Publication
(10) Pub. No.: US 2023/0225849 A1
(43) Pub. Date: Jul. 20, 2023

(54) HEART VALVE PREP ASSIST PACKAGING

(71) Applicant: BOSTON SCIENTIFIC SCIMED, INC., MAPLE GROVE, MN (US)

(72) Inventors: Johnele Chapman, Mound, MN (US); Jacob D. Keleny, Janesville, WI (US)

(73) Assignee: BOSTON SCIENTIFIC SCIMED, INC., MAPLE GROVE, MN (US)

(21) Appl. No.: 18/099,013

(22) Filed: Jan. 19, 2023

Related U.S. Application Data
(60) Provisional application No. 63/301,144, filed on Jan. 20, 2022.

(51) Int. Cl. A61F 2/00; A61F 2/24

(52) U.S. Cl. CPC A61F 2/0095; A61F 2/2427

(57) ABSTRACT

A tray assembly may include a bottom portion having a bottom wall and first, second, third, and fourth side walls each extending upward from the bottom wall, and a top portion having a top wall and first, second, third, and fourth sides. The top portion is configured to releasably engage the bottom portion. The bottom portion includes at least one notch formed in an upper edge of the first side wall, the at least one notch being configured to receive an elongate shaft of an implant delivery device. A kit for preparing a replacement heart valve implant for delivery may include the tray assembly, at least one rinsing bowl, and a loading tools kit for attaching the replacement heart valve implant to the implant delivery device.

### CROSS-REFERENCE TO RELATED APPLICATIONS

This application claims the benefit of priority of U.S. Provisional Application No. 63/301,144 filed Jan. 20, 2022, the entire disclosure of which is hereby incorporated by reference.

### TECHNICAL FIELD

The present disclosure pertains to medical devices, systems, and methods for manufacturing and/or using medical devices and/or systems. More particularly, the present disclosure pertains to packaging designed and/or configured to assist in preparing a replacement heart valve implant for use in an implantation procedure.

### BACKGROUND

Percutaneously delivered tissue-based replacement heart valve implants are typically packaged in a container that may or may not be filled with a storage solution. The storage solution may be designed to maintain the biological integrity of the implant (e.g., implant functionality, sterility, and functional integrity) while stored and awaiting use. When needed for implanting in a patient, the container is opened, and the replacement heart valve is removed using a variety of techniques aimed at preventing damage to the replacement heart valve. The replacement heart valve is then rinsed and/or immersed in fluid to prepare it for use. Next, the replacement heart valve is attached to a delivery device that will facilitate delivery of the replacement heart valve to the appropriate location in the patient's body. Typically, packaging is thrown away and a series of sterile bowls must be obtained separately to facilitate preparation of the replacement heart valve. Some replacement heart valves may have unique size, shape, and/or preparation requirements that necessitate nonstandard bowls for the prep process (e.g., must be special ordered and/or stocked by the medical facility/group and/or are only usable for a particular purpose or with a particular device/implant). Additionally, the preparation process may include steps and/or movements that place the replacement heart valve implant and/or the delivery device at risk of damage, or of falling or being dropped, which may lead to the implant and/or delivery device becoming unsterile. There is an ongoing need for alternative devices and/or methods for preparing replacement heart valve implants.

### SUMMARY

In one example, a tray assembly may comprise a bottom portion having a bottom wall and a first side wall, a second side wall, a third side wall, and a fourth side wall each extending upward from the bottom wall; and a top portion having a top wall and a first side, a second side, a third side, and a fourth side. The top portion may be configured to releasably engage the bottom portion. The bottom portion may include at least one notch formed in an upper edge of the first side wall, the at least one notch being configured to receive an elongate shaft of an implant delivery device.

In addition or alternatively to any example described herein, the bottom portion includes at least one tab extending outward from the third side wall.

In addition or alternatively to any example described herein, the first side wall is disposed opposite the third side wall and the second side wall is disposed opposite the fourth side wall.

In addition or alternatively to any example described herein, the bottom portion is sized and configured to hold about 3 liters of fluid.

In addition or alternatively to any example described herein, the top portion includes a first nesting portion extending from the top wall and a second nesting portion extending from the top wall.

In addition or alternatively to any example described herein, the first nesting portion and the second nesting portion extend downward from the top wall when the top portion is engaged with the bottom portion.

In addition or alternatively to any example described herein, the first nesting portion includes a first recess configured to receive a first end of a handle of the implant delivery device and the second nesting portion includes a second recess configured to receive a second end of the handle of the implant delivery device.

In addition or alternatively to any example described herein, the first nesting portion is disposed adjacent the first side and the second side of the top portion, and the second nesting portion is disposed adjacent the second side and the third side of the top portion.

In addition or alternatively to any example described herein, the top portion includes at least one tab extending outward from the third side.

In addition or alternatively to any example described herein, a kit for preparing a replacement heart valve implant for delivery may comprise a tray assembly, at least one rinsing bowl, and a loading tools kit for attaching the replacement heart valve implant to an implant delivery device. The tray assembly may comprise a bottom portion having a bottom wall and a first side wall, a second side wall, a third side wall, and a fourth side wall each extending upward from the bottom wall; and a top portion having a top wall and a first side, a second side, a third side, and a fourth side. The top portion may be configured to releasably engage the bottom portion. The bottom portion may include at least one notch formed in an upper edge of the first side wall, the at least one notch being configured to receive an elongate shaft of an implant delivery device.

In addition or alternatively to any example described herein, the at least one rinsing bowl and the loading tools kit nest inside of the tray assembly with the top portion engaged with the bottom portion.

In addition or alternatively to any example described herein, the at least one rinsing bowl includes three rinsing bowls.

In addition or alternatively to any example described herein, each of the at least one rinsing bowl is configured to hold about 500 milliliters of fluid.

In addition or alternatively to any example described herein, when the top portion is engaged with the bottom portion the first side of the top portion is engaged with the first side wall of the bottom portion, the second side of the top portion is engaged with the second side wall of the bottom portion, the third side of the top portion is engaged with the third side wall of the bottom portion, and the fourth side of the top portion is engaged with the fourth side wall of the bottom portion.

In addition or alternatively to any example described herein, a method of preparing a replacement heart valve implant for delivery to a patient may comprise:

opening a kit comprising:
a tray assembly comprising a bottom portion having a bottom wall and a first side wall, a second side wall, a third side wall, and a fourth side wall each extending upward from the bottom wall; and a top portion having a top wall and a first side, a second side, a third side, and a fourth side; wherein the top portion is configured to releasably engage the bottom portion;
at least one rinsing bowl; and
a loading tools kit for attaching the replacement heart valve implant to an implant delivery device;

arranging the top portion and the bottom portion on an upper surface of a table, wherein the top portion is disposed upside down and spaced apart from the bottom portion;

disposing a handle of an implant delivery device into the top portion such that a first end of the handle engages a first nesting portion of the top portion and a second end of the handle engages a second nesting portion of the top portion, wherein the implant delivery device includes an elongate shaft extending distally away from the second end of the handle;

disposing a distal end of the elongate shaft of the implant delivery device within the bottom portion such that the elongate shaft is disposed within a notch formed in an upper edge of the first side wall of the bottom portion;

rinsing the replacement heart valve implant within the at least one rinsing bowl; and

connecting the replacement heart valve implant to the elongate shaft of the implant delivery device.

In addition or alternatively to any example described herein, the at least one rinsing bowl includes a first rinsing bowl, a second rinsing bowl, and a third rinsing bowl, and rinsing the replacement heart valve implant further includes:

filling the first rinsing bowl, the second rinsing bowl, and the third rinsing bowl with saline solution;

placing the replacement heart valve implant into the saline solution in the first rinsing bowl;

transferring the replacement heart valve implant into the saline solution in the second rinsing bowl; and

subsequently transferring the replacement heart valve implant into the saline solution in the third rinsing bowl.

In addition or alternatively to any example described herein, the bottom portion of the tray assembly includes chilled saline solution disposed therein, and wherein connecting the replacement heart valve implant to the elongate shaft includes using the loading tools kit to connect the replacement heart valve implant to the elongate shaft while the distal end of the elongate shaft and the replacement heart valve implant are disposed in the chilled saline solution.

In addition or alternatively to any example described herein, the method may further comprise radially compressing the replacement heart valve implant.

In addition or alternatively to any example described herein, connecting the replacement heart valve implant to the elongate shaft includes manipulating one or more rotatable knobs on the handle while the first end of the handle is engaged with the first nesting portion and the second end of the handle is engaged with the second nesting portion.

In addition or alternatively to any example described herein, when the first end of the handle is engaged with the first nesting portion, the second end of the handle is engaged with the second nesting portion, and the elongate shaft is disposed within the notch formed in the upper edge of the first side wall of the bottom portion, the elongate shaft is oriented substantially parallel to the upper surface of the table from the top portion to the bottom portion.

The above summary of some embodiments, aspects, and/or examples is not intended to describe each disclosed embodiment or every implementation of the present disclosure. The figures and detailed description which follow more particularly exemplify these embodiments.

### BRIEF DESCRIPTION OF THE DRAWINGS

The disclosure may be more completely understood in consideration of the following detailed description in connection with the accompanying drawings, in which:

FIG. 1 illustrates selected aspects of a replacement heart valve implant;

FIG. 2 illustrates selected aspects of an implant delivery device usable with the replacement heart valve implant of FIG. 1;

FIGS. 3-8 illustrate selected aspects of a kit for preparation of a replacement heart valve implant; and

FIG. 9-17 illustrate selected aspects related to use of the kit for preparation of a replacement heart valve implant.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale, wherein like reference numerals indicate like elements throughout the several views. The detailed description and drawings are intended to illustrate example embodiments of the disclosure but not limit the disclosure. Those skilled in the art will recognize that the various elements described and/or shown may be arranged in various combinations and configurations without departing from the scope of the disclosure. However, in the interest of clarity and ease of understanding, every feature and/or element may not be shown in each drawing.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The term “extent” may be understood to mean the greatest measurement of a stated or identified dimension, unless the extent or dimension in question is preceded by or identified as a “minimum”, which may be understood to mean the smallest measurement of the stated or identified dimension. For example, “outer extent” may be understood to mean an outer dimension, “radial extent” may be understood to mean a radial dimension, “longitudinal extent” may be understood to mean a longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered the greatest possible dimension measured according to the intended usage, while a “minimum extent” may be considered the smallest possible dimension measured according to the intended usage. In some instances, an “extent” may generally be measured orthogonally within a plane and/or cross-section, but may be, as will be apparent from the particular context, measured differently—such as, but not limited to, angularly, radially, circumferentially (e.g., along an arc), etc.

The terms “monolithic” and “unitary” shall generally refer to an element or elements made from or consisting of a single structure or base unit/element. A monolithic and/or unitary element shall exclude structure and/or features made by assembling or otherwise joining multiple discrete structures or elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to use the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For example, a reference to some features may be equally referred to all instances and quantities beyond one of said feature(s) unless clearly stated to the contrary. As such, it will be understood that the following discussion may apply equally to any and/or all of the components for which there are more than one within the device, etc. unless explicitly stated to the contrary.

Additionally, it should be noted that in any given figure, some features may not be shown, or may be shown schematically, for clarity and/or simplicity. Additional details regarding some components and/or method steps may be illustrated in other figures in greater detail. The devices and/or methods disclosed herein may provide a number of desirable features and benefits as described in more detail below.

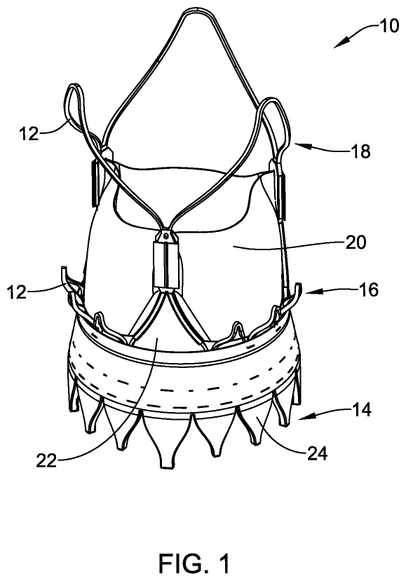

FIG. 1 illustrates selected aspects of a replacement heart valve implant 10. It should be appreciated that the replacement heart valve implant 10 can be any type of heart valve (e.g., a mitral valve, an aortic valve, etc.). In use, the replacement heart valve implant 10 may be implanted (e.g., surgically or through transcatheter delivery) in a mammalian heart. The replacement heart valve implant 10 can be configured to allow one-way flow through the replacement heart valve implant 10 from an inflow end to an outflow end.

The replacement heart valve implant 10 may include an expandable framework 12 defining a central lumen. In some embodiments, the expandable framework 12 may have a substantially circular cross-section. In some embodiments, the expandable framework 12 can have a non-circular (e.g., D-shaped, elliptical, etc.) cross-section. Some suitable but non-limiting examples of materials that may be used to form the expandable framework 12, including but not limited to metals and metal alloys, composites, ceramics, polymers, and the like, are described below. The expandable framework 12 may be configured to shift from a collapsed configuration to an expanded configuration. In some embodiments, the expandable framework 12 may be self-expanding. In some embodiments, the expandable framework 12 may be self-biased toward the expanded configuration. In some embodiments, the expandable framework 12 may be mechanically expandable. In some embodiments, the expandable framework 12 may be balloon expandable. Other configurations are also contemplated. In some embodiments, the expandable framework 12 may include and/or define a plurality of interstices (e.g., openings) through the expandable framework 12.

In some embodiments, the expandable framework 12 may define a lower crown 14 proximate an inflow end, an upper crown 16 proximate an outflow end, and a plurality of stabilization arches 18 extending downstream from the outflow end. In some embodiments, the lower crown 14 may be disposed at the inflow end. In some embodiments, the upper crown 16 may be disposed at the outflow end. In some embodiments, the plurality of stabilization arches 18 may extend downstream of and/or away from the upper crown 16 in a direction opposite the lower crown 14. In some embodiments, the upper crown 16 may be disposed longitudinally and/or axially between the lower crown 14 and the plurality of stabilization arches 18.

In some embodiments, the replacement heart valve implant 10 may include a plurality of valve leaflets 20 disposed within the central lumen. The plurality of valve leaflets 20 may be coupled, secured, and/or fixedly attached to the expandable framework 12. Each of the plurality of valve leaflets 20 may include a root edge coupled to the expandable framework 12 and a free edge (e.g., a coaptation edge) movable relative to the root edge to coapt with the free edges of the other leaflets along a coaptation region. In some embodiments, the plurality of valve leaflets 20 can be integrally formed with each other, such that the plurality of valve leaflets 20 is formed as a single unitary and/or monolithic unit. In some embodiments, the plurality of valve leaflets 20 may be formed integrally with other structures such as an inner skirt 22 and/or an outer skirt 24, base structures, liners, or the like.

The plurality of valve leaflets 20 may be configured to substantially restrict fluid from flowing through the replacement heart valve implant 10 in a closed position. For example, in some embodiments, the free edges of the plurality of valve leaflets 20 may move into coaptation with one another in the closed position to substantially restrict fluid from flowing through the replacement heart valve implant 10. The free edges of the plurality of valve leaflets 20 may be move apart from each other in an open position to permit fluid flow through the replacement heart valve implant 10. In FIG. 1, the plurality of valve leaflets 20 is shown in the open position or in a partially open position (e.g., a neutral position) that the plurality of valve leaflets 20 may move to when unbiased by fluid flow.

In some embodiments, the plurality of valve leaflets 20 may be comprised of a polymer, such as a thermoplastic polymer. In some embodiments, the plurality of valve leaflets 20 may include at least 50 percent by weight of a polymer. In some embodiments, the plurality of valve leaflets 20 may be formed from bovine pericardial or other living tissue. Other configurations and/or materials are also contemplated.

In some embodiments, the replacement heart valve implant 10 may include an inner skirt 22 disposed on and/or extending along an inner surface of the expandable framework 12. In at least some embodiments, the inner skirt 22 may be fixedly attached to the expandable framework 12. The inner skirt 22 may direct fluid, such as blood, flowing through the replacement heart valve implant 10 toward the plurality of valve leaflets 20. In at least some embodiments, the inner skirt 22 may be fixedly attached to and/or integrally formed with the plurality of valve leaflets 20. The inner skirt 22 may ensure the fluid flows through the central lumen of the replacement heart valve implant 10 and does not flow around the plurality of valve leaflets 20 when they are in the closed position.

In some embodiments, the replacement heart valve implant 10 can include an outer skirt 24 disposed on and/or extending along an outer surface of the expandable framework 12. In some embodiments, the outer skirt 24 may be disposed at and/or adjacent the lower crown 14. In some embodiments, the outer skirt 24 may be disposed between the expandable framework 12 and the vessel wall in order to prevent fluid, such as blood, flowing around the replacement heart valve implant 10 and/or the expandable framework 12 in a downstream direction. The outer skirt 24 may ensure the fluid flows through the replacement heart valve implant 10 and does not flow around the replacement heart valve implant 10, so as to ensure that the plurality of valve leaflets 20 can stop the flow of fluid when in the closed position.

In some embodiments, the inner skirt 22 may include a polymer, such as a thermoplastic polymer. In some embodiments, the inner skirt 22 may include at least 50 percent by weight of a polymer. In some embodiments, the outer skirt 24 may include a polymer, such as a thermoplastic polymer. In some embodiments, the outer skirt 24 may include at least 50 percent by weight of a polymer. In some embodiments one or more of the plurality of valve leaflets 20, the inner skirt 22, and/or the outer skirt 24 may be formed of the same polymer or polymers. In some embodiments, the polymer may be a polyurethane. In some embodiments, the inner skirt 22 and/or the outer skirt 24 may be substantially impervious to fluid. In some embodiments, the inner skirt 22 and/or the outer skirt 24 may be formed from a thin tissue (e.g., bovine pericardial, etc.). In some embodiments, the inner skirt 22 and/or the outer skirt 24 may be formed from a coated fabric material. In some embodiments, the inner skirt 22 and/or the outer skirt 24 may be formed from a nonporous and/or impermeable fabric material. Other configurations are also contemplated. Some suitable but non-limiting examples of materials that may be used to form the inner skirt 22 and/or the outer skirt 24 including but not limited to polymers, composites, and the like, are described below.

In some embodiments, the inner skirt 22 and/or the outer skirt 24 may seal one of, some of, a plurality of, or each of the plurality of interstices formed in the expandable framework 12. In at least some embodiments, sealing the interstices may be considered to prevent fluid from flowing through the interstices of the expandable framework 12. In some embodiments, the inner skirt 22 and/or the outer skirt 24 may be attached to the expandable framework 12 and/or the plurality of frame struts using one or more methods including but not limited to tying with sutures or filaments, adhesive bonding, melt bonding, embedding or over molding, welding, etc.

In some embodiments, the expandable framework 12 and/or the replacement heart valve implant 10 may have an outer extent of about 23 millimeters (mm), about 25 mm, about 27 mm, about 30 mm, etc. in an unconstrained configuration (e.g., in the expanded configuration). In some embodiments, the expandable framework 12 and/or the replacement heart valve implant 10 may have an outer extent of about 10 mm, about 9 mm about 8 mm, about 7 mm, about 6 mm, etc. in the collapsed configuration. Other configurations are also contemplated.

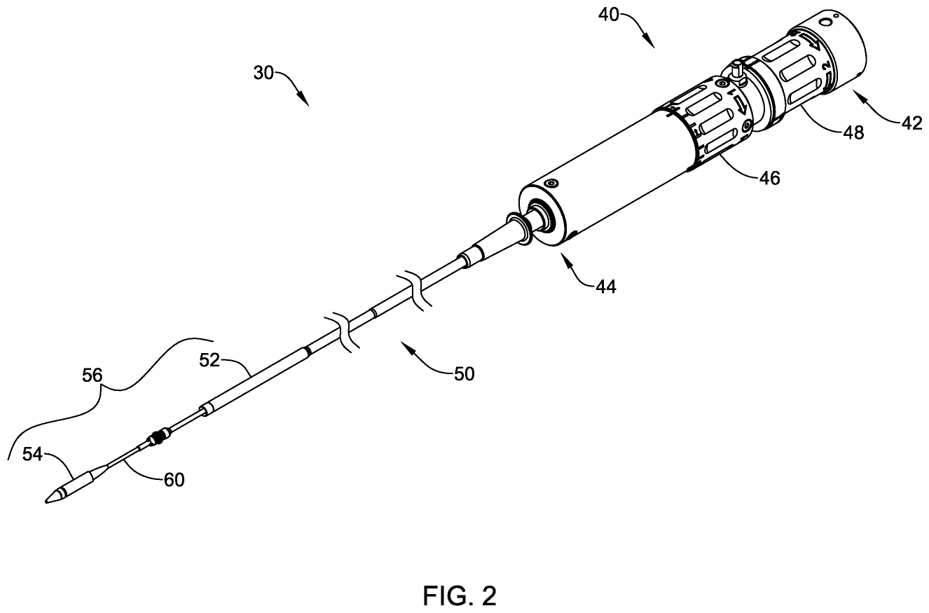

FIG. 2 illustrates selected aspects of an implant delivery device 30 compatible with and/or usable with the replacement heart valve implant 10. The implant delivery device 30 may include a handle 40 and an elongate shaft 50 extending distally from the handle 40. The handle 40 may include a first end 42 and a second end 44 opposite the first end 42. The elongate shaft 50 may extend distally from the second end 44 of the handle 40. The handle 40 may include one or more rotatable knobs. In some embodiments, the one or more rotatable knobs may include a first rotatable knob 46 and a second rotatable knob 48. In at least some embodiments, the first rotatable knob 46 and/or the second rotatable knob 48 may be configured to rotate about the central longitudinal axis of the implant delivery device 30 and/or the handle 40.

In some embodiments, the implant delivery device 30 and/or the elongate shaft 50 may include a proximal sheath 52 and a distal sheath 54. The implant delivery device 30 may include an inner shaft 60 slidably disposed within a lumen of the elongate shaft 50. The inner shaft 60 may be fixedly attached to the distal sheath 54. In some embodiments, the inner shaft 60 may include a guidewire lumen extending therethrough. In some embodiments, the handle 40 may be configured to manipulate and/or translate the proximal sheath 52 and/or the distal sheath 54 relative to each other. In some embodiments, the first rotatable knob 46 and/or the second rotatable knob 48 may be configured to manipulate and/or axially translate the proximal sheath 52 and/or the distal sheath 54 relative to each other. In some embodiments, the handle 40 may be configured to manipulate and/or translate the inner shaft 60 relative to the elongate shaft 50 and/or the proximal sheath 52. In some embodiments, the first rotatable knob 46 and/or the second rotatable knob 48 may be configured to manipulate and/or axially translate the inner shaft 60 relative to the elongate shaft 50 and/or the proximal sheath 52.

During delivery of the replacement heart valve implant 10 to a treatment site, the replacement heart valve implant 10 may be disposed within the proximal sheath 52 and/or the distal sheath 54 in a collapsed configuration. In some embodiments, the proximal sheath 52 and/or the distal sheath 54 may collectively define a stent holding portion 56 of the implant delivery device 30. In some embodiments, the stent holding portion 56 may be configured to constrain the replacement heart valve implant 10 in the collapsed configuration. In some embodiments, the replacement heart valve implant 10 may be releasably coupled to the inner shaft 60.

In use, the implant delivery device 30 may be advanced percutaneously through the vasculature to a position adjacent to the treatment site. For example, the implant delivery device 30 may be advanced through the vasculature and across the aortic arch to a position adjacent to a defective native heart valve. Alternative approaches to treat a defective aortic valve and/or other heart valve(s) are also contemplated with the implant delivery device 30. After navigating the implant delivery device 30 and/or the stent holding portion 56 to the treatment site, the proximal sheath 52 and/or the distal sheath 54 may be translated relative to each other to open the stent holding portion 56. When unconstrained by the stent holding portion 56, the replacement heart valve implant 10 may be configured to shift from the collapsed configuration to an expanded configuration. In at least some interventions, the replacement heart valve implant 10 may be deployed within the native heart valve (e.g., the native heart valve is left in place and not excised). Alternatively, the native heart valve may be removed (such as through valvuloplasty, for example) and the replacement heart valve implant 10 may be deployed in its place as a replacement. Some suitable but non-limiting materials for the implant delivery device 30, the handle 40, the elongate shaft 50, the proximal sheath 52, the distal sheath 54, the inner shaft 60, and/or components or elements thereof, for example metallic materials and/or polymeric materials, are described below.

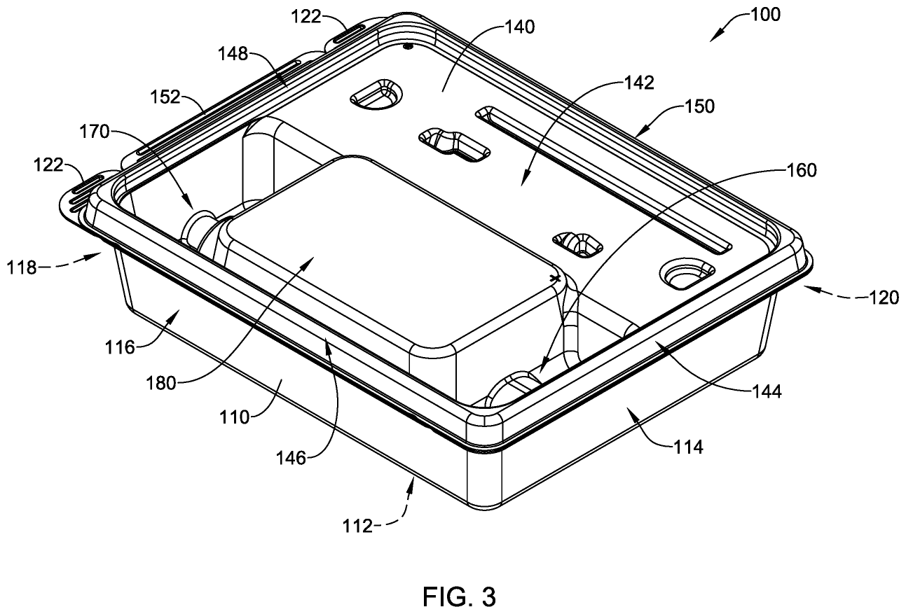

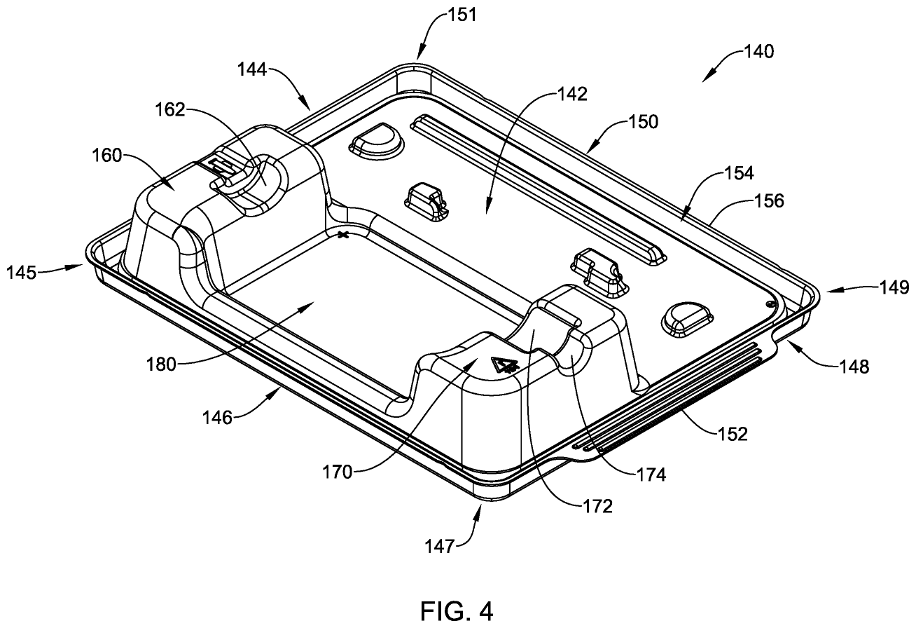

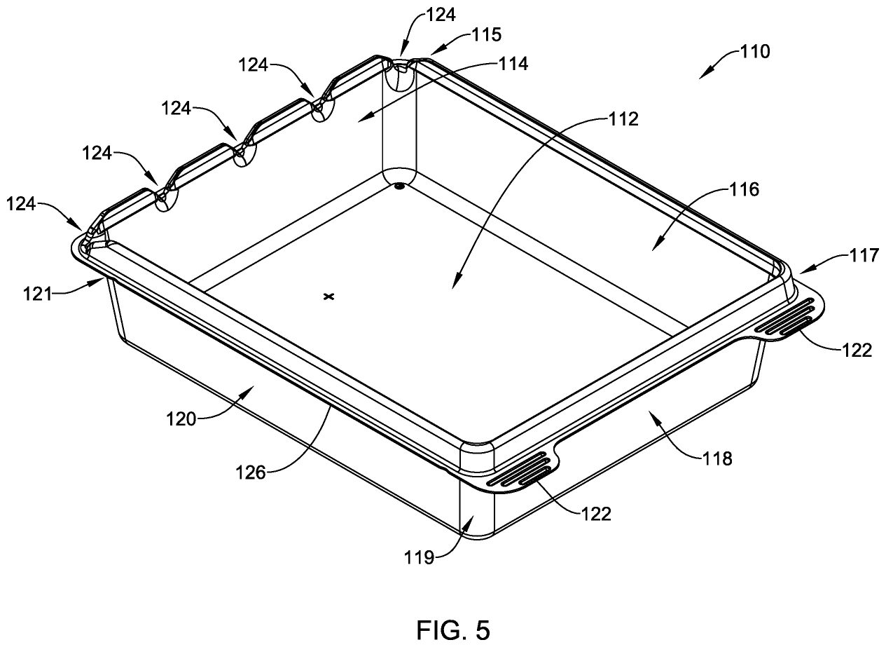

FIGS. 3-5 illustrate selected aspects of a tray assembly 100 associated with the disclosure. Some features and/or details may be shown in more than one figure and/or in other figures and/or views for clarity. The tray assembly 100 (e.g., FIG. 3) may include a bottom portion 110 (e.g., FIGS. 3 and 5) and a top portion 140 (e.g., FIGS. 3 and 4—note that in FIG. 4, the top portion 140 is shown “upside down”). The top portion 140 may be configured to releasably engage and/or mate with the bottom portion 110. The top portion 140 may be independent of and/or distinct from the bottom portion 110. For reasons that will become apparent, the top portion 140 must be able to be completely separated from the bottom portion 110. As such, the top portion 140 may not be permanently attached to the bottom portion 110 when the top portion 140 is engaged with the bottom portion 110. In at least some embodiments, there is no hinge mechanism between and/or connecting the top portion 140 and the bottom portion 110. In some embodiments, the tray assembly 100 is devoid of a hinge mechanism between and/or connecting the top portion 140 and the bottom portion 110. In some embodiments, there is no frangible connection between the top portion 140 and the bottom portion 110. In some embodiments, the tray assembly 100 is devoid of a frangible connection between the top portion 140 and the bottom portion 110.

In some embodiments, the bottom portion 110 may have and/or include a bottom wall 112 and a first side wall 114, a second side wall 116, a third side wall 118, and a fourth side wall 120 each extending upward from the bottom wall 112. Other configurations are also contemplated. For example, in some embodiments, the bottom portion 110 may have and/or include more or less than four side walls (e.g., three side walls, five side walls, six side walls, etc.). In some embodiments, the first side wall 114 may be disposed opposite the third side wall 118 and the second side wall 116 may be disposed opposite the fourth side wall 120.

The bottom portion 110 may include and/or define a first corner 115 at an intersection of the first side wall 114 and the second side wall 116. The bottom portion 110 may include and/or define a second corner 117 at an intersection of the second side wall 116 and the third side wall 118. The bottom portion 110 may include and/or define a third corner 119 at an intersection of the third side wall 118 and the fourth side wall 120. The bottom portion 110 may include and/or define a fourth corner 121 at an intersection of the fourth side wall 120 and the first side wall 114.

In some embodiments, the bottom portion 110 may include at least one notch 124 formed along and/or in an upper edge of the first side wall 114. In some embodiments, the at least one notch 124 may extend downwardly and/or toward the bottom wall 112 from the upper edge of the first side wall 114. In some embodiments, each of the at least one notch 124 may form a recess, an indent, etc. in the first side wall 114 and/or in the upper edge of the first side wall 114. In some embodiments, the at least one notch 124 may be disposed along the first side wall 114 between the first corner 115 of the bottom portion 110 and the fourth corner 121 of the bottom portion 110. In some embodiments, the at least one notch 124 may be formed at the first corner and/or at the fourth corner and/or between the first corner and the fourth corner. Other configurations are also contemplated.

In some embodiments, the at least one notch 124 may be sized and configured to receive the elongate shaft 50 of the implant delivery device 30. In some embodiments, one of, some of, or each of the at least one notch 124 may be generally V-shaped. In some embodiments, one of, some of, or each of the at least one notch 124 include a generally V-shaped upper portion and a curved lower portion. In some embodiments, one of, some of, or each of the at least one notch 124 include a generally V-shaped upper portion and a generally U-shaped lower portion. Other configurations are also contemplated.

In some embodiments, the bottom portion 110 may include at least one tab 122 extending outward from the third side wall 118. In some embodiments, the at least one tab 122 may extend outward from an upper portion of the third side wall 118. In some embodiments, the bottom portion 110 may include a lip 126 extending downward and outward from an upper edge of the first side wall 114, the second side wall 116, the third side wall 118, and the fourth side wall 120. In some embodiments, the lip 126 may extend continuously around a perimeter of the bottom portion 110. In some embodiments, the lip 126 may extend discontinuously around the perimeter of the bottom portion 110. In some embodiments, the at least one tab 122 may extend outward from the lip 126.

In some embodiments, the at least one tab 122 may include a first tab and a second tab. In some embodiments, the first tab may be disposed at and/or adjacent the second corner 117 of the bottom portion 110. In some embodiments, the second tab may be disposed at and/or adjacent the third corner 119 of the bottom portion 110. In some embodiments, the first tab and/or the second tab may be disposed between the second corner 117 and the third corner 119. Other configurations are also contemplated. In some embodiments, the first tab and/or the second tab may extend outward from the lip 126.

In some embodiments, the bottom portion 110 may be sized and configured to hold about 3 liters of fluid, such as saline solution or another sterile fluid. In some embodiments, the bottom portion 110 may be sized and configured to hold more or less than 3 liters of fluid. Other configurations are also contemplated. In some embodiments, the bottom portion 110 may be sized and configured to hold chilled saline solution, as described herein. In some embodiments, the chilled saline solution may include saline solution in combination with a cooling element (e.g., sterile ice chips, a frozen sterile saline bag, etc.). Other configurations are also contemplated. The bottom portion 110 may also be sized and configured to organize, hold, and/or retain elements of a kit for preparing a replacement heart valve implant for delivery, as described herein.

In some embodiments, the bottom portion 110 may have a maximum longitudinal extent (e.g., length) of about 12.50 inches to about 13.00 inches. In some embodiments, the bottom portion 110 may have a maximum longitudinal extent (e.g., length) of about 12.88 inches. Other configurations and/or sizes are also contemplated. In some embodiments, the maximum longitudinal extent may include the at least one tab 122. In some embodiments, the bottom portion 110 may have a maximum lateral extent (e.g., width) of about 9.75 inches to about 10.25 inches. In some embodiments, the bottom portion 110 may have a maximum lateral extent (e.g., width) of about 9.96 inches. Other configurations and/or sizes are also contemplated. In some embodiments, the bottom portion 110 may be about 2.50 inches to about 3.00 inches deep. In some embodiments, the bottom portion 110 may be about 2.89 inches deep. Other configurations and/or sizes are also contemplated. In some embodiments, the bottom portion 110 may have an interior length at the upper edges of the first side wall 114 and the third side wall 118 of about 11.00 inches to about 11.50 inches. In some embodiments, the bottom portion 110 may have an interior length at the upper edges of the first side wall 114 and the third side wall 118 of about 11.33 inches. Other configurations and/or sizes are also contemplated. In some embodiments, the bottom portion 110 may have an interior width at the upper edges of the second side wall 116 and the fourth side wall 120 of about 8.75 inches to about 9.25 inches. In some embodiments, the bottom portion 110 may have an interior length at the upper edges of the second side wall 116 and the fourth side wall 120 of about 9.06 inches. Other configurations and/or sizes are also contemplated.

The top portion 140 may have and/or include a top wall 142 and a first side 144, a second side 146, a third side 148, and a fourth side 150. Other configurations are also contemplated. For example, in some embodiments, the top portion 140 may have and/or include more or less than four sides (e.g., three sides, five sides, six sides, etc.). In some embodiments, the first side 144 may be disposed opposite the third side 148 and the second side 146 may be disposed opposite the fourth side 150.

The top portion 140 may include and/or define a first corner 145 at an intersection of the first side 144 and the second side 146. The top portion 140 may include and/or define a second corner 147 at an intersection of the second side 146 and the third side 148. The top portion 140 may include and/or define a third corner 149 at an intersection of the third side 148 and the fourth side 150. The top portion 140 may include and/or define a fourth corner 151 at an intersection of the fourth side 150 and the first side 144.

In some embodiments, the top portion 140 may include at least one tab 152 extending outward from the third side 148. In some embodiments, the at least one tab 152 may be disposed along the third side 148. In some embodiments, the at least one tab 152 may be disposed between the second corner 147 and the third corner 149 of the top portion 140. In some embodiments, the at least one tab 152 may extend along a majority of the third side 148. In some embodiments, the at least one tab 152 of the top portion 140 may be offset laterally (e.g., toward the second side wall 116 and/or the fourth side wall 120) from the at least one tab 122 of the bottom portion 110 when the top portion 140 is engaged with the bottom portion 110. In some embodiments, the at least one tab 152 of the top portion 140 may be disposed between the first tab and the second tab of the bottom portion 110 when the top portion 140 is engaged with the bottom portion 110.

In some embodiments, the top portion 140 may include a lip 156 extending downward and outward from the first side 144, the second side 146, the third side 148, and the fourth side 150. As shown in FIG. 4, where the top portion 140 is illustrated “upside down”, the lip 156 extends upward and outward from the first side 144, the second side 146, the third side 148, and the fourth side 150. In some embodiments, the lip 156 may extend continuously around a perimeter of the top portion 140. In some embodiments, the lip 156 may extend discontinuously around the perimeter of the top portion 140. In some embodiments, the at least one tab 152 may extend outward from the lip.

In some embodiments, the top portion 140 may define and/or include a channel 154 disposed between and/or at least partially defined by the top wall 142 and the lip 156. The channel 154 of the top portion 140 may be configured to receive and/or engage the upper edge(s) of the first side wall 114, the second side wall 116, the third side wall 118, and the fourth side wall 120 of the bottom portion 110. In at least some embodiments, the lip 156 of the top portion 140 may be configured to engage, contact, and/or abut the lip 126 of the bottom portion 110 when the top portion 140 is engaged with the bottom portion 110.

In some embodiments, the top portion 140 may include a first nesting portion 160 formed in and/or extending from the top wall 142. In at least some embodiments, the first nesting portion 160 may be monolithically formed with the top wall 142. In some embodiments, the top portion 140 may include a second nesting portion 170 formed in and/or extending from the top wall 142. In at least some embodiments, the second nesting portion 170 may be monolithically formed with the top wall 142. In at least some embodiments, the top portion 140 may include the first nesting portion 160 and the second nesting portion 170. The second nesting portion 170 may be spaced apart from the first nesting portion 160. In some embodiments, the first nesting portion 160 may extend downward from the top wall 142 when the top portion 140 is engaged with the bottom portion 110, as seen in FIG. 3 for example. As shown in FIG. 4, where the top portion 140 is illustrated “upside down”, the first nesting portion 160 may extend upward from the top portion 140. In some embodiments, the second nesting portion 170 may extend downward from the top wall 142 when the top portion 140 is engaged with the bottom portion 110, as seen in FIG. 3 for example. As shown in FIG. 4, where the top portion 140 is illustrated “upside down”, the second nesting portion 170 may extend upward from the top wall 142.

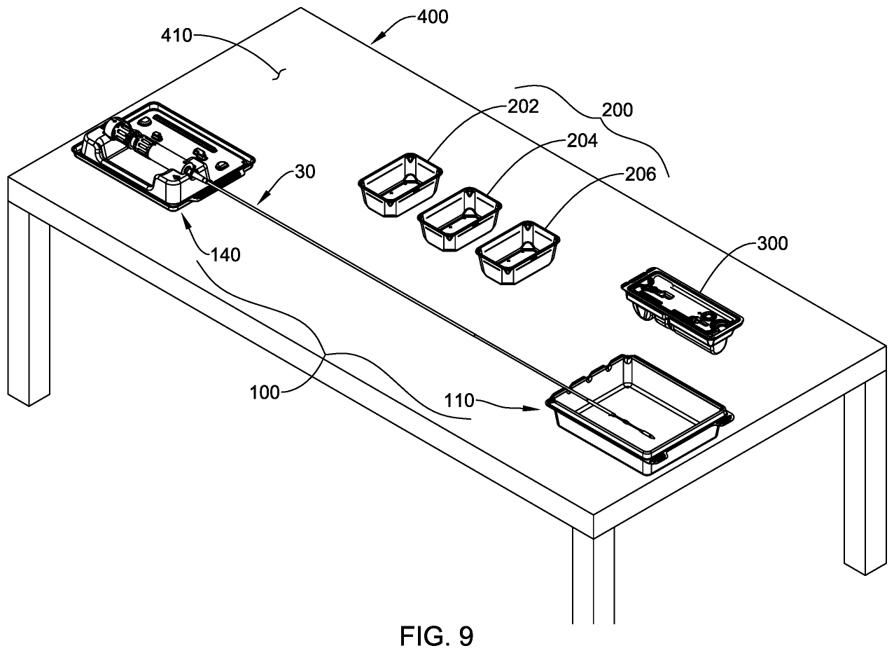

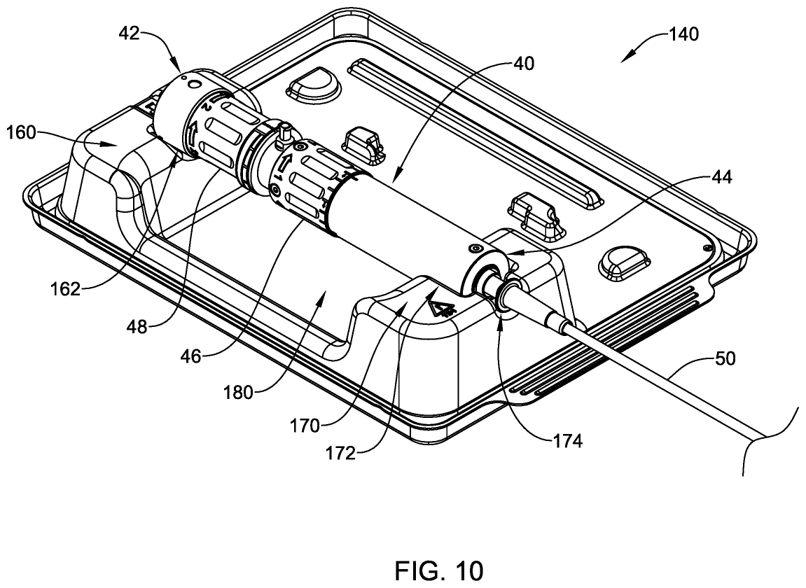

In some embodiments, the first nesting portion 160 may include a first recess 162 sized and configured to receive the first end 42 of the handle 40 of the implant delivery device 30 (e.g., FIGS. 9-10). In some embodiments, the second nesting portion 170 may include a second recess 172 sized and configured to receive the second end 44 of the handle 40 of the implant delivery device 30 (e.g., FIGS. 9-10). In some embodiments, the second nesting portion 170 may include a third recess 174 sized and configured to receive the elongate shaft 50 of the implant delivery device 30 (e.g., FIGS. 9-10).

As seen in FIGS. 3-4, the first nesting portion 160 may be disposed at and/or adjacent the first corner 145 of the top portion 140. In some embodiments, the first nesting portion 160 may be disposed adjacent the first side 144 and/or the second side 146 of the top portion 140. The second nesting portion 170 may be disposed at and/or adjacent the second corner 147 of the top portion 140. In some embodiments, the second nesting portion 170 may be disposed adjacent the second side 146 and/or the third side 148 of the top portion 140. Other configurations are also contemplated.

In some embodiments, the top portion 140 may include a third nesting portion 180 formed in and/or extending from the top wall 142. In at least some embodiments, the third nesting portion 180 may be monolithically formed with the top wall 142. In some embodiments, the third nesting portion 180 may be disposed between the first nesting portion 160 and the second nesting portion 170. In some embodiments, the third nesting portion 180 may be immediately adjacent to the first nesting portion 160 and/or the second nesting portion 170. In some embodiments, the first nesting portion 160 may form at least a part of the third nesting portion 180. In some embodiments, the first nesting portion 160 may form a first end of the third nesting portion 180. In some embodiments, the second nesting portion 170 may form at least a part of the third nesting portion 180. In some embodiments, the second nesting portion 170 may form a second end of the third nesting portion 180 opposite the first end of the third nesting portion 180. In some embodiments, when the top portion 140 is engaged with the bottom portion 110, the third nesting portion 180 may extend upward from the top wall 142. As shown in FIG. 4, where the top portion 140 is illustrated “upside down”, the third nesting portion 180 may extend downward from the top wall 142. In some embodiments, the third nesting portion 180 may include a flattened portion oriented generally parallel to the top wall 142. The flattened portion may extend from the first nesting portion 160 toward and/or to the second nesting portion 170.

Returning briefly to FIG. 3, when the top portion 140 is engaged with the bottom portion 110, the first side 144 of the top portion 140 may be engaged with the first side wall 114 of the bottom portion 110. When the top portion 140 is engaged with the bottom portion 110, the second side 146 of the top portion 140 may be engaged with the second side wall 116 of the bottom portion 110. When the top portion 140 is engaged with the bottom portion 110, the third side 148 of the top portion 140 may be engaged with the third side wall 118 of the bottom portion 110. When the top portion 140 is engaged with the bottom portion 110, the fourth side 150 of the top portion 140 may be engaged with the fourth side wall 120 of the bottom portion 110. In some embodiments, the at least one tab 122 of the bottom portion 110 may be disposed and/or positioned adjacent to the at least one tab 152 of the top portion 140 such that the at least one tab 122 of the bottom portion 110 and the at least one tab 152 of the top portion 140 may be pulled apart to separate, detach, and/or disengage the top portion 140 from the bottom portion 110.

The tray assembly 100, the bottom portion 110, and/or the top portion 140 may be manufactured using one or more of a variety of manufacturing methods. In one example, the tray assembly 100, the bottom portion 110, and/or the top portion 140 may be injection molded. In another example, the tray assembly 100, the bottom portion 110, and/or the top portion 140 may be blow molded, rotomolded, vacuum molded, or another suitable method of molding. In some embodiments, the tray assembly 100, the bottom portion 110, and/or the top portion 140 may be cast, machined, laser cut, stamped, etc. Other methods of production are also contemplated. Some suitable but non-limiting examples of materials that may be used to for the tray assembly 100, the bottom portion 110, and/or the top portion 140, including but not limited to metals and metal alloys, composites, ceramics, polymers, and the like, are described below.

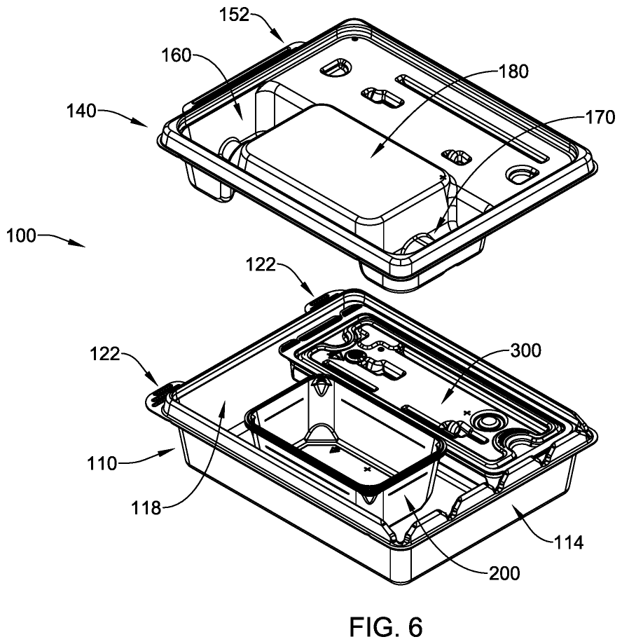

FIG. 6 illustrates selected aspects related to a kit for preparing the replacement heart valve implant 10 for delivery. In some embodiments, the kit may include the tray assembly 100, as described herein. In FIG. 6, the top portion 140 has been separated, detached, and/or disengaged from the bottom portion 110 to reveal contents and/or components of the kit. In some embodiments, the kit may include at least one rinsing bowl 200. In some embodiments, the kit may include a loading tools kit 300 for attaching the replacement heart valve implant 10 to the implant delivery device 30. In at least some embodiments, the loading tools kit 300 may be specific to the replacement heart valve implant 10. In some embodiments, the loading tools kit 300 may include generic tools and/or components for use with any replacement heart valve implant. Other configurations are also contemplated. In some embodiments, the at least one rinsing bowl 200 and the loading tools kit 300 nest inside of the tray assembly 100 with the top portion 140 engaged with the bottom portion 110.

In some embodiments, the third nesting portion 180 may be sized and configured to receive a top portion of the at least one rinsing bowl 200 when the top portion 140 is engaged with the bottom portion 110. In some embodiments, the third nesting portion 180 may be sized and configured to receive a bottom portion of the at least one rinsing bowl 200 when the top portion 140 is engaged with the bottom portion 110. In some embodiments, the first nesting portion 160 and the second nesting portion 170 may cooperate with the third nesting portion 180 to position and/or maintain the at least one rinsing bowl 200 generally centered between the first side wall 114 and the third side wall 118 of the bottom portion 110 when the top portion 140 is engaged with the bottom portion 110 and the at least one rinsing bowl 200 is disposed within the tray assembly 100.

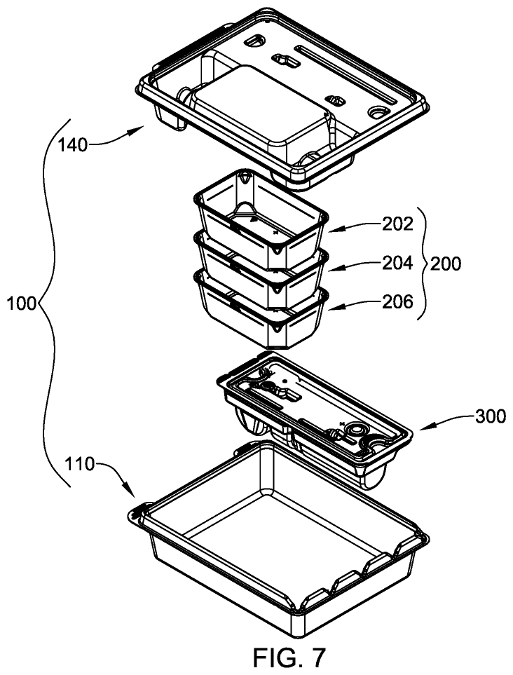

In some embodiments, the at least one rinsing bowl 200 may include a plurality of rinsing bowls. In some embodiments, the at least one rinsing bowl 200 may include three rinsing bowls. In some embodiments, the at least one rinsing bowl 200 may include a first rinsing bowl 202, a second rinsing bowl 204, and a third rinsing bowl 206, as shown in FIG. 7. Other configurations are also contemplated. For example, in some embodiments, the kit and/or the at least one rinsing bowl 200 may include more or less than three rinsing bowls (e.g., two rinsing bowls, four rinsing bowls, five rinsing bowls, etc.). In some embodiments, if the kit is specific to the replacement heart valve implant 10, the at least one rinsing bowl 200 may include three rinsing bowls, as described herein. In some embodiments, if the kit is generic to any replacement heart valve implant, the at least one rinsing bowl 200 may include more or less than three rinsing bowls.

In some embodiments, each of the at least one rinsing bowl 200 (e.g., the first rinsing bowl 202, the second rinsing bowl 204, and the third rinsing bowl 206) may be shaped and configured to nest inside of each other. In some embodiments, each of the at least one rinsing bowl 200 (e.g., the first rinsing bowl 202, the second rinsing bowl 204, and the third rinsing bowl 206) may be shaped and configured to nest inside of each other inside of the tray assembly 100 and/or the bottom portion 110 of the tray assembly 100. In some embodiments, each of the at least one rinsing bowl 200 (e.g., the first rinsing bowl 202, the second rinsing bowl 204, and the third rinsing bowl 206) may be sized and configured to hold about 500 milliliters of fluid.

In some embodiments, each of the at least one rinsing bowl 200 (e.g., the first rinsing bowl 202, the second rinsing bowl 204, and the third rinsing bowl 206) may include a fill line on at least one side designating a level corresponding to 500 milliliters of fluid. Other configurations are also contemplated. In some embodiments, each of the at least one rinsing bowl 200 (e.g., the first rinsing bowl 202, the second rinsing bowl 204, and the third rinsing bowl 206) may include a fill line on at least one side designating a level corresponding to an amount greater than 500 milliliters of fluid (e.g., 550 milliliters, 750 milliliters, 1 liter, etc.). In some embodiments, each of the at least one rinsing bowl 200 (e.g., the first rinsing bowl 202, the second rinsing bowl 204, and the third rinsing bowl 206) may include a fill line on at least one side designating a level corresponding to an amount less than 500 milliliters of fluid (e.g., 450 milliliters, 250 milliliters, etc. In some embodiments, each of the at least one rinsing bowl 200 (e.g., the first rinsing bowl 202, the second rinsing bowl 204, and the third rinsing bowl 206) may include multiple fill lines designating different levels of fluid contained therein.

The at least one rinsing bowl 200 (e.g., the first rinsing bowl 202, the second rinsing bowl 204, and the third rinsing bowl 206) may be manufactured using one or more of a variety of manufacturing methods. In one example, the at least one rinsing bowl 200 (e.g., the first rinsing bowl 202, the second rinsing bowl 204, and the third rinsing bowl 206) may be injection molded. In another example, the at least one rinsing bowl 200 (e.g., the first rinsing bowl 202, the second rinsing bowl 204, and the third rinsing bowl 206) may be blow molded, rotomolded, vacuum molded, or another suitable method of molding. In some embodiments, the at least one rinsing bowl 200 (e.g., the first rinsing bowl 202, the second rinsing bowl 204, and the third rinsing bowl 206) may be cast, machined, laser cut, stamped, etc. Other methods of production are also contemplated. Some suitable but non-limiting examples of materials that may be used to for the at least one rinsing bowl 200 (e.g., the first rinsing bowl 202, the second rinsing bowl 204, and the third rinsing bowl 206), including but not limited to metals and metal alloys, composites, ceramics, polymers, and the like, are described below.

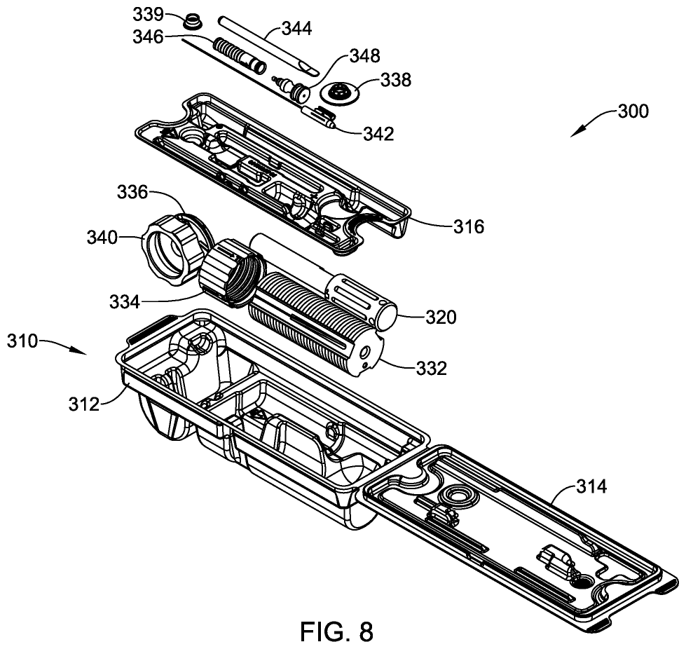

FIG. 8 illustrates selected aspects of the loading tools kit 300. The loading tools kit 300 may be designed and configured for use in attaching the replacement heart valve implant 10 to the elongate shaft 50 of the implant delivery device 30. In at least some embodiments, the loading tools kit 300 may be configured for use in attaching the replacement heart valve implant 10 to the stent holding portion 56 of the elongate shaft 50 of the implant delivery device 30.

In some embodiments, the loading tools kit 300 may include a tool tray assembly 310 having a bottom portion 312 and a top portion 314 hingedly attached to the bottom portion 312. In some embodiments, the top portion 314 may be detachable from the bottom portion 312 (e.g., the top portion 314 may be a distinct and/or separate piece from the bottom portion 312). In some embodiments, the bottom portion 312 may be configured to hold and/or organize some larger components of the loading tools kit 300 and an inner tray 316 configured to hold and/or organize some smaller components of the loading tools kit 300. The inner tray 316 may be configured to nest inside of the bottom portion 312 of the tool tray assembly 310.

In some embodiments, the loading tools kit 300 may include a proximal loader 320 configured to attach a proximal portion of the replacement heart valve implant 10 and/or the expandable framework 12 to the elongate shaft 50, and/or to adjust and/or finalize a position of the replacement heart valve implant 10 and/or the expandable framework 12 relative to the elongate shaft 50 while the replacement heart valve implant 10 is being attached to the elongate shaft 50 of the implant delivery device 30. In some embodiments, the loading tools kit 300 may include a distal loader configured to radially compress the replacement heart valve implant 10 and/or the expandable framework 12 prior to attaching the expandable framework 12 to the elongate shaft 50 of the implant delivery device 30. In some embodiments, the distal loader may include one or more of a loading funnel 332, a first threaded member 334, a star pusher 336, an iris 338, a compressing ring 339, and a second threaded member 340. In some embodiments, the distal loader may include other and/or additional components. In some embodiments, the loading funnel 332, the first threaded member 334, and the star pusher 336 may be used to radially compress the replacement heart valve implant 10 from the expanded configuration to the compressed configuration. In some embodiments, the iris 338, the compressing ring 339, and the second threaded member 340 may be configured to engage the loading funnel 332 and/or the compressed replacement heart valve implant 10 to attach the expandable framework 12 to the elongate shaft 50 of the implant delivery device 30.

A basic level of explanation of selected features of the distal loader is now provided merely for reference. In some embodiments, the loading funnel 332 may include external threads configured to engage the first threaded member 334 and/or the second threaded member 340. In some embodiments, the loading funnel 332 may be injection molded, cast, or machined. Other configurations and/or methods of production are also contemplated. In some embodiments, the loading funnel 332 may be formed as a monolithic structure from a single piece of material. In some embodiments, the loading funnel 332 may be formed from two or more individual pieces that are then assembled together using one or more known techniques. In some embodiments, the loading funnel 332 may include an inner surface having a taper. In some embodiments, the inner surface of the loading funnel 332 may taper radially inward toward the distal end of the loading funnel 332.

In some embodiments, the star pusher 336 may be configured to translate axially along the loading funnel 332. The first threaded member 334 may be configured to rotatably and/or threadably engage the loading funnel 332. In some embodiments, the loading funnel 332 may include external threads configured to receive internal threads formed in the first threaded member 334. The first threaded member 334 may be configured to rotate relative to the loading funnel 332. The first threaded member 334 may be configured to translate axially relative to the loading funnel 332. In at least some embodiments, rotation of the first threaded member 334 relative to the loading funnel 332 when the first threaded member 334 is engaged with the loading funnel 332 may translate the first threaded member 334 axially along and/or relative to the loading funnel 332. In some embodiments, the first threaded member 334 may be configured to engage and translate the star pusher 336 axially along and/or relative to the loading funnel 332 as the first threaded member 334 is rotated relative to the loading funnel 332.

The star pusher 336 may include an outer ring and a plurality of legs extending radially inward from the outer ring. The plurality of legs may be configured to be disposed in and/or to project through a plurality of longitudinal slots formed through a side wall of the loading funnel 332. As such, the star pusher 336 may be non-rotatable relative to the loading funnel 332 when the star pusher 336 is engaged with the loading funnel 332. The star pusher 336 may be adapted and/or configured to push and/or translate a stent and/or the replacement heart valve implant 10 axially along, relative to, and/or through the loading funnel 332 as the star pusher 336 is translated axially along and/or relative to the loading funnel 332.

The iris 338 may be configured to be positioned adjacent the distal end of the loading funnel 332. The iris 338 may include a plurality of arms extending parallel to the central longitudinal axis and defining a central opening. Other configurations are also contemplated. In some embodiments, each of the plurality of arms of the iris 338 may include a tapered outer surface. In at least some embodiments, the plurality of arms may be configured to resiliently flex, deflect, and/or bend to permit relative movement between adjacent arms of the plurality of arms. In some embodiments, the iris 338 may be manufactured using one or more a variety of methods. In some embodiments, the iris 338 may be machined, injection molded, cast, and/or cut using a waterjet or laser. Other methods of manufacture are also contemplated.

The second threaded member 340 may be configured to rotatably and/or threadably engage the distal end of the loading funnel 332 and the plurality of arms of the iris 338. In some embodiments, rotation of the second threaded member 340 relative to the loading funnel 332 may change a size of the central opening of the iris 338. In some embodiments, the plurality of arms of the iris 338 may be configured to deflect radially between a first configuration and a second configuration upon rotation of the second threaded member 340 relative to the loading funnel 332. In at least some embodiments, the plurality of arms of the iris 338 may be self-biased toward the first configuration.

In some embodiments, the loading tools kit 300 may include a stylet 342 configured to be inserted into the handle 40 of the implant delivery device 30 during loading of the replacement heart valve implant 10. In some embodiments, the loading tools kit 300 may include a spatula 344 configured to fold the plurality of valve leaflets 20 after the replacement heart valve implant 10 has been compressed using the distal loader. In some embodiments, the loading tools kit 300 may include a loading tube 346 configured to hold the plurality of stabilization arches 18 in the collapsed configuration, after the expandable framework 12 has been attached to the elongate shaft 50, while the plurality of stabilization arches 18 is captured within the proximal sheath 52. In some embodiments, the loading tools kit 300 may include a safety button 348 configured to engage the handle 40 during flushing of the implant delivery device 30, after attachment of and before delivery of the replacement heart valve implant 10. Other and/or additional tools may be provided as needed.

Some suitable but non-limiting examples of materials that may be used to for the loading funnel 332, the first threaded member 334, the star pusher 336, the iris 338, the compressing ring 339, the second threaded member 340, the stylet 342, the spatula 344, the loading tube 346, the safety button 348, etc., including but not limited to metals and metal alloys, composites, ceramics, polymers, and the like, are described below.

FIG. 9-17 illustrate selected aspects related to use of the kit for preparation of the replacement heart valve implant 10 and/or a method of preparing the replacement heart valve implant 10 for delivery to the patient. In at least some embodiments, the method may include opening the kit, as described herein. In some embodiments, the kit for preparation of the replacement heart valve implant 10 may be shipped sterile in a bag or package. In some embodiments, the bag or package may be configured to permit sterilization of the kit within the bag or package. In some embodiments, the kit may be sterilized before it is placed into the bag or package. After opening the kit, the bag or package may be disposed of according to established procedures (e.g., hospital/facility practice or policy, etc.).

In some embodiments, the method may include arranging the top portion 140 of the tray assembly 100 and the bottom portion 110 of the tray assembly 100 on an upper surface 410 of a table 400, as shown in FIG. 9. In at least some embodiments, the table 400 may be disposed within an operating room at a hospital or clinic. In some embodiments, the table 400 may be disposed in a preparatory room adjacent to and/or near the operating room. Other configurations are also contemplated. The upper surface 410 of the table 400 and/or the whole table 400 may be sanitized and/or sterilized prior to use. In at least some embodiments, the top portion 140 may be disposed upside down and spaced apart from the bottom portion 110. In some embodiments, the at least one notch 124 formed in the upper edge of the first side wall 114 of the bottom portion 110 may be positioned and/or oriented toward the top portion 140 on the table 400. In some embodiments, the at least one tab 152 of the top portion 140 may extend toward the bottom portion 110 on the table 400.

In some embodiments, the first side wall 114 of the bottom portion 110 and the first side 144 of the top portion 140 may face in a common direction, a single direction, the same direction, etc. on and/or relative to the table 400 (e.g., both may face to the left, as viewed in FIG. 9). The second side wall 116 of the bottom portion 110 and the second side 146 of the top portion 140 may face in a common direction, a single direction, the same direction, etc. on and/or relative to the table 400. The third side wall 118 of the bottom portion 110 and the third side 148 of the top portion 140 may face in a common direction, a single direction, the same direction, etc. on and/or relative to the table 400. The fourth side wall 120 of the bottom portion 110 and the fourth side 150 of the top portion 140 may face in a common direction, a single direction, the same direction, etc. on and/or relative to the table 400.

In some embodiments, the method may include arranging the at least one rinsing bowl 200 (e.g., the first rinsing bowl 202, the second rinsing bowl 204, the third rinsing bowl 206, etc.) on the upper surface 410 of the table 400 near the top portion 140 and/or the bottom portion 110. In FIG. 9, the at least one rinsing bowl 200 (e.g., the first rinsing bowl 202, the second rinsing bowl 204, the third rinsing bowl 206, etc.) is disposed, arranged, and/or positioned generally equidistant from and/or generally centered between the top portion 140 and the bottom portion 110, but this is not required. In some embodiments, the at least one rinsing bowl 200 (e.g., the first rinsing bowl 202, the second rinsing bowl 204, the third rinsing bowl 206, etc.) may be disposed, arranged, and/or positioned closer to the bottom portion 110 than the top portion 140. Other configurations are also contemplated.

In some embodiments, the method may include disposing the loading tools kit 300 near and/or adjacent to the bottom portion 110 of the tray assembly 100. In some embodiments, the method may include opening the tool tray assembly 310 of the loading tools kit 300. In some embodiments, the method may include removing the inner tray 316 from the bottom portion 312 of the tool tray assembly 310. In some embodiments, the method may include removing one or more loading tools and/or components thereof from the inner tray 316 and/or the bottom portion 312 of the tool tray assembly 310. In some embodiments, the method may include arranging the one or more loading tools and/or components removed from the inner tray 316 and/or the bottom portion 312 of the tool tray assembly 310 on the upper surface 410 of the table 400. In some embodiments, at least some of the loading tools and/or components thereof may remain in and/or may be left in the inner tray 316 and/or the bottom portion 312 of the tool tray assembly 310 until needed for use.

The method may include disposing the handle 40 of the implant delivery device 30 into the top portion 140 of the tray assembly 100, as seen in FIG. 9 and in more detail in FIG. 10, such that the first end 42 of the handle 40 engages the first nesting portion 160 of the top portion 140 and the second end 44 of the handle 40 engages the second nesting portion 170 of the top portion 140. The first end 42 of the handle 40 may be disposed in and/or may engage the first recess 162 formed in the first nesting portion 160. The second end 44 of the handle 40 may be disposed in and/or may engage the second recess 172 formed in the second nesting portion 170. The elongate shaft 50 may extend distally away from the second end 44 of the handle 40. The elongate shaft 50 may be disposed in and/or may engage the third recess 174 formed in the second nesting portion 170.

As shown clearly in FIG. 10, the third nesting portion 180 may extend between the first nesting portion 160 and the second nesting portion 170. At least some of the handle 40 may extend over and/or may be positioned above the third nesting portion 180 such that a user's hands may access the handle 40 adjacent the third nesting portion 180. The one or more rotatable knobs (e.g., the first rotatable knob 46 and/or the second rotatable knob 48) may be disposed over and/or above the third nesting portion 180. A user may be able to position his or her hand(s) on the handle 40 and/or around the handle 40 adjacent to the third nesting portion 180 and/or between the first nesting portion 160 and the second nesting portion 170 to access, engage, and/or manipulate the one or more rotatable knobs (e.g., the first rotatable knob 46 and/or the second rotatable knob 48). This configuration of the top portion 140 (e.g., engagement of the handle 40 with the top portion 140) may permit access to and/or manipulation of the handle 40 while limiting risk of the handle 40 rolling and/or falling off of the table 400. The top portion 140 and/or engagement of the handle 40 with the top portion 140 may also limit risk of the handle 40 rolling and/or falling off of the table 400 as the elongate shaft 50 (e.g., the stent holding portion 56, etc.) is accessed and/or manipulated to connect and/or attach the replacement heart valve implant 10 thereto.

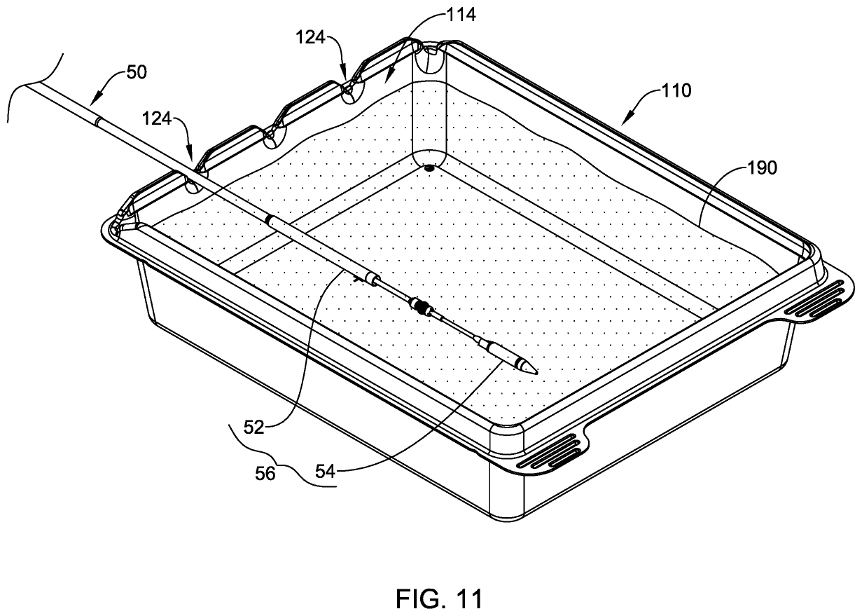

The method may include disposing the distal end (e.g., the proximal sheath 52, the distal sheath 54, the stent holding portion 56, etc.) of the elongate shaft 50 of the implant delivery device 30 within the bottom portion 110 of the tray assembly 100, as seen in FIG. 9 and in more detail in FIG. 11, such that the elongate shaft 50 is disposed within a notch (of the at least one notch 124) formed in the upper edge of the first side wall 114 of the bottom portion 110.

In some embodiments, the bottom portion 110 of the tray assembly 100 may include chilled saline solution 190 disposed therein. In at least some embodiments, the distal end (e.g., the proximal sheath 52, the distal sheath 54, the stent holding portion 56, etc.) of the elongate shaft 50 of the implant delivery device 30 may be disposed within and/or may be submerged in the chilled saline solution 190 in the bottom portion 110 of the tray assembly 100 to facilitate flushing and/or purging of air from the implant delivery device 30. For example, after flushing the implant delivery device 30, maintaining unsealed access points into the implant delivery device 30 within the chilled saline solution 190 may prevent and/or limit air ingress into the implant delivery device 30. In at least some embodiments, it may be important to maintain the replacement heart valve implant 10 submerged in fluid as much as possible to preserve and/or maintain the integrity of the plurality of valve leaflets 20, the inner skirt 22, the outer skirt 24, etc.

In some embodiments, the top portion 140 and the bottom portion 110 may be sized and configured to hold and/or position the implant delivery device 30 and/or the elongate shaft 50 of the implant delivery device 30 substantially parallel to the upper surface 410 of the table 400. In some embodiments, when the first end 42 of the handle 40 is engaged with the first nesting portion 160, the second end 44 of the handle 40 is engaged with the second nesting portion 170, and the elongate shaft 50 is disposed within the notch (of the at least one notch 124) formed in the upper edge of the first side wall 114 of the bottom portion 110, as seen in FIG. 9 for example, the elongate shaft 50 may be oriented substantially parallel to the upper surface 410 of the table 400 from the top portion 140 to the bottom portion 110.

Alternatively, in some embodiments, the top portion 140 and the bottom portion 110 may be sized and configured to hold and/or position the implant delivery device 30 and/or the elongate shaft 50 of the implant delivery device 30 at an acute angle relative to the upper surface 410 of the table 400. In some embodiments, when the first end 42 of the handle 40 is engaged with the first nesting portion 160, the second end 44 of the handle 40 is engaged with the second nesting portion 170, and the elongate shaft 50 is disposed within the notch (of the at least one notch 124) formed in the upper edge of the first side wall 114 of the bottom portion 110, as seen in FIG. 9 for example, the elongate shaft 50 may be oriented at an acute angle relative to the upper surface 410 of the table 400 from the top portion 140 to the bottom portion 110. In some embodiments, the acute angle may be less than 30 degrees, less than 20 degrees, less than 10 degrees, less than 5 degrees, etc. For example, in some embodiments, when the first end 42 of the handle 40 is engaged with the first nesting portion 160, the second end 44 of the handle 40 is engaged with the second nesting portion 170, and the elongate shaft 50 is disposed within the notch (of the at least one notch 124) formed in the upper edge of the first side wall 114 of the bottom portion 110, the distal end of the elongate shaft 50 may be disposed closer to the upper surface 410 of the table 400 than a proximal end of the elongate shaft 50. Other configurations are also contemplated.

In some embodiments, the method may include rinsing the replacement heart valve implant 10 within the at least one rinsing bowl 200. In some embodiments, the at least one rinsing bowl 200 may include a first rinsing bowl 202, a second rinsing bowl 204, and a third rinsing bowl 206. In some embodiments, rinsing the replacement heart valve implant 10 may include filling each of the first rinsing bowl 202, the second rinsing bowl 204, and the third rinsing bowl 206 with saline solution 210. In some embodiments, the saline solution 210 may be pre-chilled. In some embodiments, the saline solution 210 may be room temperature. Other configurations are also contemplated. In some embodiments, the third rinsing bowl 206 may further include a predetermined amount of a therapeutic agent (e.g., heparin, etc.) disposed therein and/or mixed into the saline solution 210. Some suitable but non-limiting examples of therapeutic agents are described below.

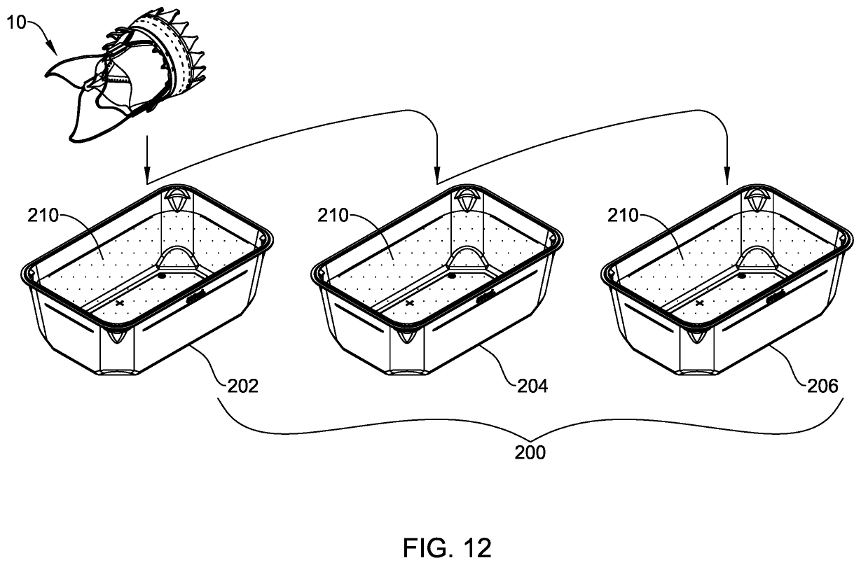

In some embodiments, the method and/or rinsing the replacement heart valve implant 10 may include placing the replacement heart valve implant 10 into the saline solution 210 in the first rinsing bowl 202, transferring the replacement heart valve implant 10 into the saline solution 210 in the second rinsing bowl 204, and subsequently transferring the replacement heart valve implant 10 into the saline solution 210 in the third rinsing bowl 206, as shown in FIG. 12.

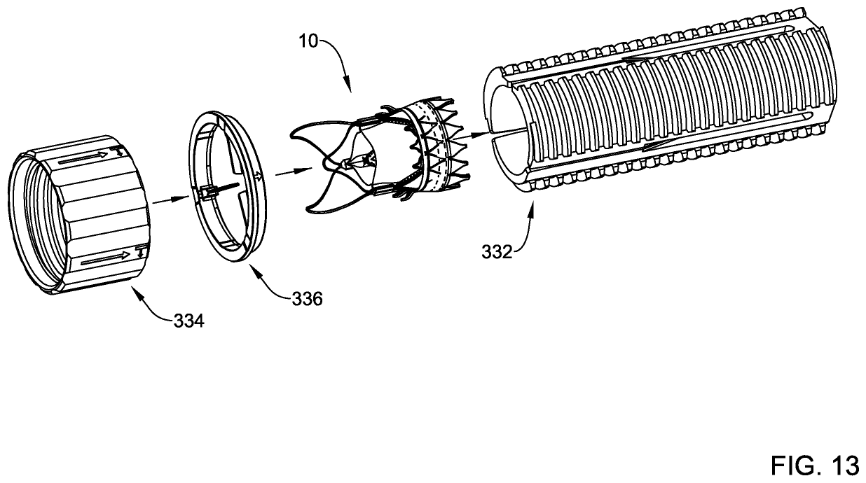

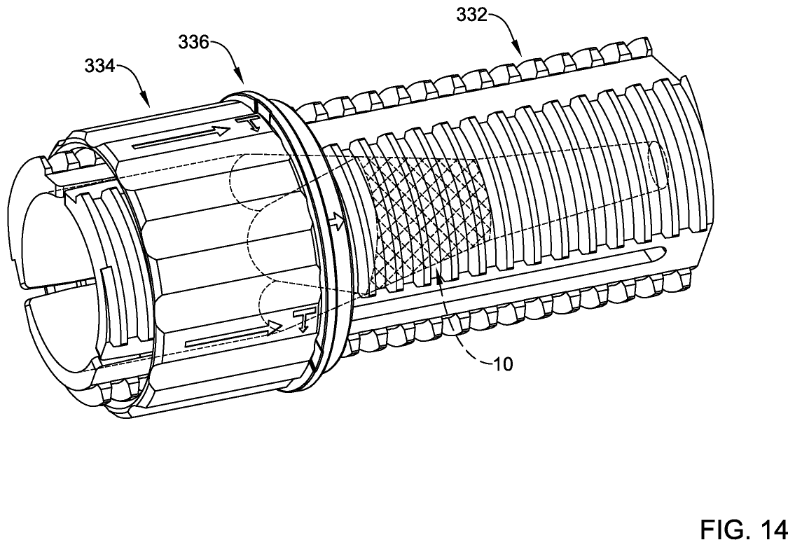

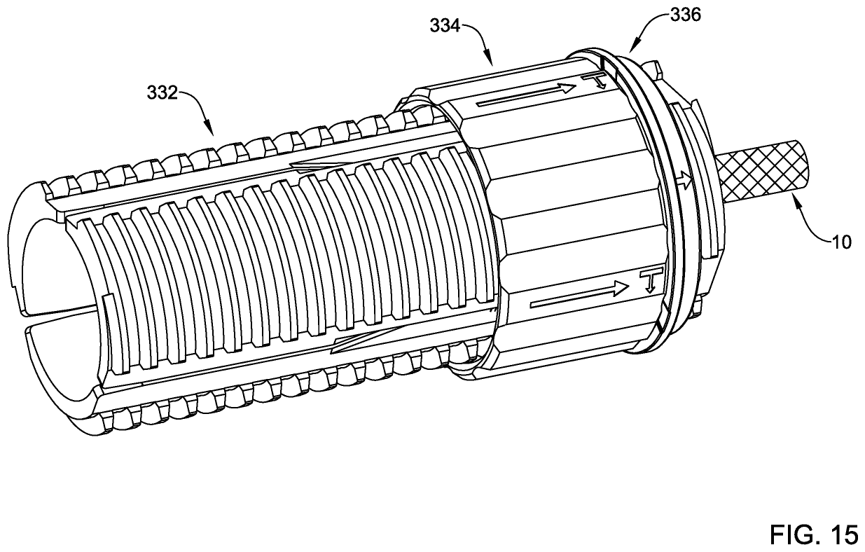

In some embodiments, the method may comprise radially compressing the replacement heart valve implant 10, as illustrates schematically in FIGS. 13-15. In some embodiments, the loading tools kit 300 (e.g., FIG. 8) and/or components thereof may be used to radially compress the replacement heart valve implant 10. For example, the distal loader may be used to radially compress the replacement heart valve implant 10. In some embodiments, the replacement heart valve implant 10 may be inserted into a first end of the loading funnel 332 and followed by the star pusher 336, as seen in FIG. 13. The first threaded member 334 may be threaded onto the first end of the loading funnel 332 and advanced toward a second end of the loading funnel 332 opposite the first end, thereby advancing the replacement heart valve implant 10 through the loading funnel 332, as seen in FIG. 14. As the replacement heart valve implant 10 exits the second end of the loading funnel 332, the replacement heart valve implant 10 may be disposed in the compressed configuration, as seen in FIG. 15.

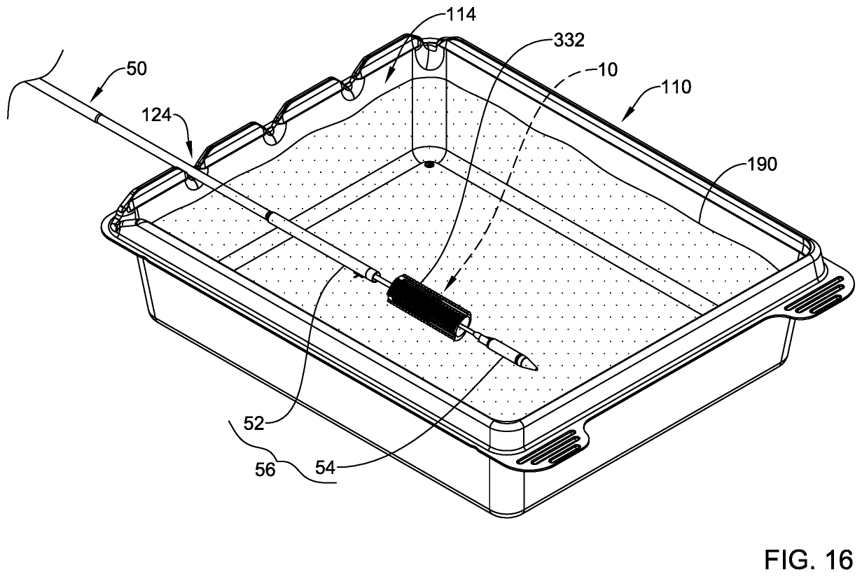

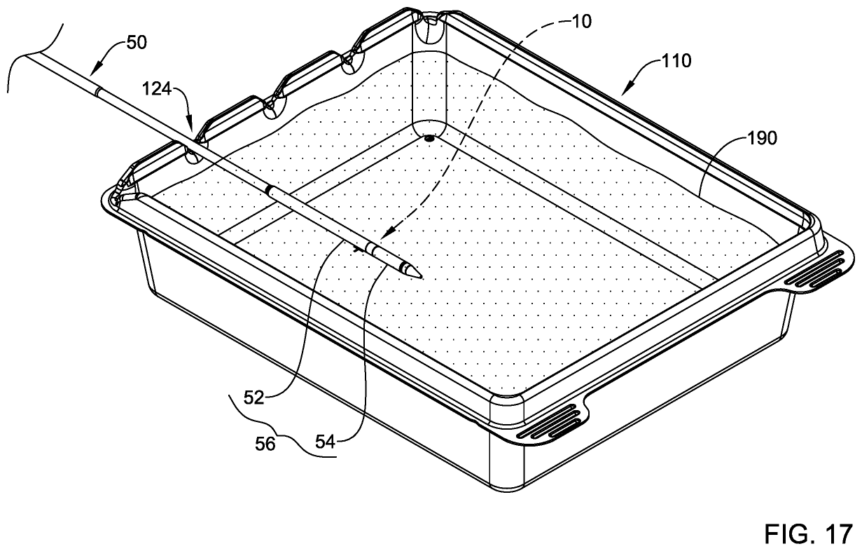

In some embodiments, the method may include connecting the replacement heart valve implant 10 to the elongate shaft 50 of the implant delivery device 30. In some embodiments, connecting the replacement heart valve implant 10 to the elongate shaft 50 may include using the loading tools kit 300 and/or components thereof to connect the replacement heart valve implant 10 to the elongate shaft 50 while the distal end of the elongate shaft 50 (e.g., the stent holding portion 56) and the replacement heart valve implant 10 are disposed in the chilled saline solution 190 disposed within the bottom portion 110 of the tray assembly 100, as shown schematically in FIGS. 16-17.

In some embodiments, the elongate shaft 50 and/or the inner shaft 60 may be positioned within and/or may extend through the distal loader and/or the loading funnel 332 while the replacement heart valve implant 10 is radially compressed using the distal loader, as seen schematically in FIG. 16. In some embodiments, once a distal end or an inflow end of the replacement heart valve implant 10 and/or the expandable framework 12 is connected to and/or attached to the elongate shaft 50 (e.g., disposed within the distal sheath 54, for example), the loading funnel 332 may be translated proximally along the elongate shaft 50 and/or the inner shaft 60 and the loading tube 346 may be advanced proximally over the replacement heart valve implant 10 (at the same time as the loading funnel 332) to hold and/or maintain the plurality of stabilization arches 18 in the compressed configuration while the proximal sheath 52 is advanced distally relative to the replacement heart valve implant 10 to cover and/or capture the plurality of stabilization arches 18 therein, as seen schematically in FIG. 17.

In at least some embodiments, connecting the replacement heart valve implant 10 to the elongate shaft 50 may include manipulating one or more rotatable knobs (e.g., the first rotatable knob 46 and/or the second rotatable knob 48, etc.) on the handle 40 while the first end 42 of the handle 40 is engaged with the first nesting portion 160 and the second end 44 of the handle 40 is engaged with the second nesting portion 170. Space around the handle 40 provided by the first nesting portion 160, the second nesting portion 170, and the third nesting portion 180 may permit a user's hand(s) to access and/or manipulate the one or more rotatable knobs (e.g., the first rotatable knob 46 and/or the second rotatable knob 48, etc.) on the handle 40.

The materials that can be used for the various components of the kit and the various elements thereof disclosed herein may include those commonly associated with medical devices and/or packaging used and/or associated with medical devices. For simplicity purposes, the following discussion refers to the kit. However, this is not intended to limit the devices, components, and methods described herein, as the discussion may be applied to other elements, members, components, or devices disclosed herein, such as, but not limited to, the tray assembly, the at least one rinsing bowl, the loading tools kit, etc. and/or elements or components thereof.

In some embodiments, the kit and/or components thereof may be made from a metal, metal alloy, polymer (some examples of which are disclosed below), a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM, for example, DELRIN®), polyether block ester, polyurethane (for example, Polyurethane 85A), polypropylene (PP), polyvinylchloride (PVC), polyether-ester (for example, ARNITEL®), ether or ester based copolymers (for example, butylene/poly(alkylene ether) phthalate and/or other polyester elastomers such as HYTREL®), polyamide (for example, DURETHAN® or CRISTAMID®), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID®), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, polyisobutylene (PIB), polyisobutylene polyurethane (PIBU), polyurethane silicone copolymers (for example, Elast-Eon® or ChronoSil®), ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In some embodiments, the kit and/or other elements disclosed herein may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethyl ketone)); antiproliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl ketone, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); cholesterol-lowering agents; vasodilating agents; and agents which interfere with endogenous vasoactive mechanisms.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. A tray assembly, comprising:
a bottom portion having a bottom wall and a first side wall, a second side wall, a third side wall, and a fourth side wall each extending upward from the bottom wall; and
a top portion having a top wall and a first side, a second side, a third side, and a fourth side;
wherein the top portion is configured to releasably engage the bottom portion;
wherein the bottom portion includes at least one notch formed in an upper edge of the first side wall, the at least one notch being configured to receive an elongate shaft of an implant delivery device.

2. The tray assembly of claim 1, wherein the bottom portion includes at least one tab extending outward from the third side wall.

3. The tray assembly of claim 1, wherein the first side wall is disposed opposite the third side wall and the second side wall is disposed opposite the fourth side wall.

4. The tray assembly of claim 1, wherein the bottom portion is sized and configured to hold about 3 liters of fluid.

5. The tray assembly of claim 1, wherein the top portion includes a first nesting portion extending from the top wall and a second nesting portion extending from the top wall.

6. The tray assembly of claim 5, wherein the first nesting portion and the second nesting portion extend downward from the top wall when the top portion is engaged with the bottom portion.

7. The tray assembly of claim 5, wherein the first nesting portion includes a first recess configured to receive a first end of a handle of the implant delivery device and the second nesting portion includes a second recess configured to receive a second end of the handle of the implant delivery device.

8. The tray assembly of claim 5, wherein the first nesting portion is disposed adjacent the first side and the second side of the top portion, and the second nesting portion is disposed adjacent the second side and the third side of the top portion.

9. The tray assembly of claim 1, wherein the top portion includes at least one tab extending outward from the third side.

10. A kit for preparing a replacement heart valve implant for delivery, comprising:
a tray assembly, comprising:
a bottom portion having a bottom wall and a first side wall, a second side wall, a third side wall, and a fourth side wall each extending upward from the bottom wall; and
a top portion having a top wall and a first side, a second side, a third side, and a fourth side;
wherein the top portion is configured to releasably engage the bottom portion;
wherein the bottom portion includes at least one notch formed in an upper edge of the first side wall, the at least one notch being configured to receive an elongate shaft of an implant delivery device;
at least one rinsing bowl; and
a loading tools kit for attaching the replacement heart valve implant to an implant delivery device.

11. The kit of claim 10, wherein the at least one rinsing bowl and the loading tools kit nest inside of the tray assembly with the top portion engaged with the bottom portion.

12. The kit of claim 10, wherein the at least one rinsing bowl includes three rinsing bowls.

13. The kit of claim 10, wherein each of the at least one rinsing bowl is configured to hold about 500 milliliters of fluid.

14. The kit of claim 10, wherein when the top portion is engaged with the bottom portion:
the first side of the top portion is engaged with the first side wall of the bottom portion;
the second side of the top portion is engaged with the second side wall of the bottom portion;
the third side of the top portion is engaged with the third side wall of the bottom portion; and
the fourth side of the top portion is engaged with the fourth side wall of the bottom portion.

15. A method of preparing a replacement heart valve implant for delivery to a patient, comprising:
opening a kit comprising:
a tray assembly comprising:
a bottom portion having a bottom wall and a first side wall, a second side wall, a third side wall, and a fourth side wall each extending upward from the bottom wall; and
a top portion having a top wall and a first side, a second side, a third side, and a fourth side;
wherein the top portion is configured to releasably engage the bottom portion;
at least one rinsing bowl; and
a loading tools kit for attaching the replacement heart valve implant to an implant delivery device;
arranging the top portion and the bottom portion on an upper surface of a table, wherein the top portion is disposed upside down and spaced apart from the bottom portion;
disposing a handle of an implant delivery device into the top portion such that a first end of the handle engages a first nesting portion of the top portion and a second end of the handle engages a second nesting portion of the top portion, wherein the implant delivery device includes an elongate shaft extending distally away from the second end of the handle;
disposing a distal end of the elongate shaft of the implant delivery device within the bottom portion such that the elongate shaft is disposed within a notch formed in an upper edge of the first side wall of the bottom portion;
rinsing the replacement heart valve implant within the at least one rinsing bowl; and
connecting the replacement heart valve implant to the elongate shaft of the implant delivery device.

16. The method of claim 15, wherein the at least one rinsing bowl includes a first rinsing bowl, a second rinsing bowl, and a third rinsing bowl, and rinsing the replacement heart valve implant further includes:
filling the first rinsing bowl, the second rinsing bowl, and the third rinsing bowl with saline solution;
placing the replacement heart valve implant into the saline solution in the first rinsing bowl;
transferring the replacement heart valve implant into the saline solution in the second rinsing bowl; and
subsequently transferring the replacement heart valve implant into the saline solution in the third rinsing bowl.

17. The method of claim 15, wherein the bottom portion of the tray assembly includes chilled saline solution disposed therein, and wherein connecting the replacement heart valve implant to the elongate shaft includes using the loading tools kit to connect the replacement heart valve implant to the elongate shaft while the distal end of the elongate shaft and the replacement heart valve implant are disposed in the chilled saline solution.

18. The method of claim 15, further comprising radially compressing the replacement heart valve implant.

19. The method of claim 15, wherein connecting the replacement heart valve implant to the elongate shaft includes manipulating one or more rotatable knobs on the handle while the first end of the handle is engaged with the first nesting portion and the second end of the handle is engaged with the second nesting portion.

20. The method of claim 15, wherein when the first end of the handle is engaged with the first nesting portion, the second end of the handle is engaged with the second nesting portion, and the elongate shaft is disposed within the notch formed in the upper edge of the first side wall of the bottom portion, the elongate shaft is oriented substantially parallel to the upper surface of the table from the top portion to the bottom portion.
